(19) United States
(12) Patent Application Publication
(10) Pub. No.: US 2016/0256370 A1
(43) Pub. Date: Sep. 8, 2016

(54) HAIR RETENTIVE FORMULATION AND METHOD OF USE

(71) Applicant: Restorsea, LLC, New York, NY (US)

(72) Inventors: Enrique P. Alabata, Torrance, CA (US); Patricia S. Pao, New York, NY (US)

(73) Assignee: Restorsea, LLC, New York, NY (US)

(21) Appl. No.: 15/059,562

(22) Filed: Mar. 3, 2016

Related U.S. Application Data
(60) Provisional application No. 62/129,380, filed on Mar. 6, 2015.

(51) Int. Cl. A61K 8/34; A61B 5/00; A61K 9/00; A61M 35/00; A61Q 7/00; A61K 8/37

(52) U.S. Cl. CPC A61K 8/345; A61Q 7/00; A61K 8/375; A61K 9/0014; A61M 35/00; A61B 5/448; A61B 5/4848; A61K 2800/70; A61K 2800/596

(57) ABSTRACT

A formulation and use thereof in reducing hair loss on the scalp of a human subject, where the formulation contains at least one penetration enhancer effective in reducing hair loss when applied topically to a scalp.

### RELATED APPLICATION

This application claims priority to U.S. provisional application Ser. No. 62/129,380, filed on Mar. 6, 2015, the contents of which are incorporated herein by reference.

### FIELD OF THE INVENTION

The present invention in general relates to a formulation for promoting hair retention and a method for the use thereof.

### BACKGROUND OF THE INVENTION

There has been considerable effort throughout history to hide or minimize hair loss. Until the 20th century, an individual suffering hair loss had to resort to wigs, toupees or other accoutrements to hide the hair loss. While various plant extracts have shown some success in treating hair loss, the results can vary considerably from one individual to another. A topically applied formulation containing an extract from hatching salmon eggs is also known for use in reducing hair loss. With the discovery that minoxidil promotes hair growth and the recognition that hair loss is a biochemical process, pharmaceuticals have been developed to promote hair growth. The oral medication finasteride is a treatment option for men, but is not approved for use by women. Both minoxidil and finasteride have been known to cause occasional side effects.

Promoting hair retention and promoting hair growth are two different things, though each contributes to the overall effect of increasing the length and number of hair strands. A treatment that promotes hair growth may stimulate growth of new hair shafts from a quiescent hair follicle, or may stimulate an existing, growing hair shaft to grow more rapidly. A treatment that promotes hair retention prolongs the time that existing hair shafts remain imbedded in the scalp, prior to their being lost in the normal cycle of hair growth and shedding. Unfortunately, appropriate biological targets for compounds that will improve hair retention or hair growth remain unclear, with various possible mechanisms being implicated. Candidate targets illustratively include nerve growth factor (NGF), thymosin beta4 (TB4), heparanase, activin, and dihydrotestosterone. Given the lack of understanding of the mechanisms resulting in different types of clinically defined alopecia, there is a dearth of options for many individuals that suffer hair loss.

Thus, there exists a need for a safe and effective treatment able to promote hair retention and thereby promote a fuller head of hair.

### SUMMARY OF THE INVENTION

A method of treating a human subject to promote hair retention on the subject's scalp is provided. The method includes identifying a human subject in need of a reduction in rate of hair loss and applying topically to the scalp of the subject, at least once per week for at least four weeks, an effective amount of a formulation comprising one or more skin penetration enhancers that are effective in reducing the rate of hair loss when applied to a scalp, wherein the one or more skin penetration enhancers are the only ingredients in the formulation that are active in reducing the rate of hair loss or otherwise increasing the number of hairs on a human scalp. In some embodiments, the formulation is left in place on the subject's scalp following each application, e.g., more than 15 minutes or more than an hour, up to the time of the subject's next shampoo, which may be hours or days later; while in other embodiments, the formulation is washed out shortly after application, e.g., within 15 minutes or even within 5 minutes.

Also provided is a method of treating a human subject to promote hair retention on the subject's scalp, where the method includes identifying a human subject in need of a reduction in rate of hair loss and applying topically to the scalp of the subject, at least once every two days for at least four weeks, an effective amount of a formulation comprising an aqueous solution containing an octanediol and a hexanediol, wherein the formulation does not contain proteins from hatching eggs (such as fish eggs). In some embodiments, the formulation does not contain any lipases; in others, it does not contain any animal proteins. In some embodiments, the octanediol is 1,2-octanediol, the hexanediol is 1,2-hexanediol, and each constitutes at least 0.5 total weight percent of the formulation. The formulation may also contain 1,2-pentanediol, e.g., in an amount that is at least 5 total weight percent of the formulation. In some embodiments, the formulation is allowed to dry on the scalp after each application.

Use of a skin penetration enhancer or a combination of skin penetration enhancers as an active agent in the manufacture of a medicament for reducing rate of hair loss from a scalp is also provided, In some embodiments, the skin penetration enhancer or combination of skin penetration enhancers is the only ingredient in the formulation that is active in reducing the rate of hair loss or otherwise affecting hair density on a human scalp. The skin penetration enhancer or combination of skin penetration enhancers may include, for example, one or more C₃-C₁₆ diols.

Also provided is a skin penetration enhancer or combination of skin penetration enhancers as an active agent for use in reducing rate of hair loss from a scalp. The skin penetration enhancer or combination of skin penetration enhancers may include, for example, one or more C₃-C₁₆ diols.

Also provided is a formulation comprising, as the sole active ingredient, a skin penetration enhancer or a combination of skin penetration enhancers, for use in reducing rate of hair loss when applied topically to a scalp. The skin penetration enhancer or combination of skin penetration enhancers may include, for example, one or more C₃-C₁₆ diols.

Also provided is a product for reducing the rate of hair loss on the scalp of a hymn subject, the product comprising a vessel containing a formulation that is an aqueous solution comprising one, two or three diols selected from octanediols, hexanediols, and pentanediols, the diol(s) being present in an amount that promotes hair retention when applied to a human scalp, wherein the formulation is suitable for topical application to a human scalp and contains no protein from hatching eggs (e.g., fish eggs) and no active ingredients other than the diol(s). The product includes a device suitable for applying the formulation to a human scalp, the device being a spray pump, a sponge applicator, or an eye dropper. in some embodiments, the formulation contains at least 0.5 total weight percent 1,2-octanediol and at least 0.5 total weight percent 1,2-hexanediol,

### DETAILED DESCRIPTION OF THE INVENTION

The present invention has utility as a means to promote hair retention—i.e., to reduce the rate of hair loss. It has been surprisingly discovered that a skin penetration enhancer (or a combination of skin penetration enhancers) is effective in improving retention of hair on the scalp of a subject (i.e., reduce the number of hairs naturally shed in a given time period). The improved retention can in some subjects result in a 20% or higher (e.g., at least 25%, at least 30%, at least 40%, or at least 50%) reduction in the rate of hair loss, depending on the length of use. The percent reduction in hair loss can be quantified by, for example, collecting and counting the number of hairs shed by the subject during washing, drying and comb-out of the subject's hair at a given sitting after consistent use of the formulation for a period of time, and comparing that number to the number of similarly collected hairs shed at a point prior to beginning use of the formulation. When some formulations of the invention were tested in this manner in a plurality of human test subjects for at least 12 weeks, they were found to be effective in producing an average reduction of over 40% in the number of hairs shed by the test subjects, compared to the number of hairs shed by the test subjects at time zero. A formulation within the invention that was tested for a longer period of time produced an average reduction in hairs shed of over 50% when used by the test subjects for 20 weeks. In general, formulations within the invention will produce a 20% or greater reduction in the average number of hairs shed at a given sitting by human test subjects who use the formulation for at least 20 weeks. In some embodiments, the reduction in the average number of hairs shed after 20 weeks of use will be 25% or more, 30% or more, 40% or more, or 45% or more.

The formulation and method of usage are suitable for both male and female subjects. The method may be used over a prolonged period of time (e.g., years) to maintain the improved retention of hair and thereby increase the number of hairs on the scalp and the appearance of denser hair.

Without intending to be bound to a particular theory, a skin penetration enhancer may clean the hair follicle of accumulated sebum, dead skin cells, and dirt, and/or may solubilize metabolites associated with hair loss. The sebum, etc. and/or solubilized metabolites may then be carried away by blood perfusing the scalp capillaries or may be washed off by subsequent shampooing.

It is to be understood that, in any instance where a range of values is provided herein, the range is intended to encompass not only the two end point values of the range, but also intermediate values within the range, with the intermediate values having the same number (or fewer) of significant figures as does an explicit endpoint of the range. By way of example, a recited range of from 1 to 4 is intended, for purposes of the present disclosure only, to constitute a disclosure of 1, 2, 3, 4, 1-2, 1-3, 2-4, 3-4, and 1-4.

A skin penetration enhancer (or combination thereof) is applied to the scalp neat or in solution. Suitable skin penetration-assisting agents include sulfoxides such as dimethylsulfoxide (DMSO) azones such as 1-butyl-2-azacycloheptanones, 1-hexyl-2-aracycloheptanones, 1-octyl-2-aracycloheptanones, 1-dodecylazacycloheptan-2-one, or laurocapram; pyrrolidones such as 2-pyrrolidone, 1-ethyl-2-pyrrolidone, 1-butyl-2-pyrrolidone, 1-hexyl-2-pyrrolidone, 1-octyl-2-pyrrolidone, 1-dodecyl-2-pyrrolidone, or N-methyl-2-pyrrolidone; amides such as transhydroxyproline-N-decanamide-C-ethylamide, N,N-dimethylhexanamide, N,N-dimethylheptanamide, N,N-dimethyloctanamide, or N,N-dimethylnonanamide; piperidinones such as 1-butyl-2-piperidinone, 1-hexyl-2-piperidinone, or 1-octyl-2-piperidinone; C₃-C₁₆ alkanois and alkenois such as 1-propanol, 1-butanol, 1-pentanol, 1-hexanol, 1-heptanol, 1-octanol, 1-nonanol, 1-decanol, 1-undecanol, 3-hexanol, 2-heptanol, 3-heptanol, 4-heptanol, 2-octanol, 3-octanol, 4-octanol, 2-nonanol, cis-3-penten-1-ol, or cis-3-hexen-1-ol; C₆-C₁₆ aryl alcohols such as benzyl alcohol, 2-phenoxyethanol, or 2-phenylethanol; C₃-C₁₆ thols such as 1,2-propanediol propylene glycol), 1,2-butanediol, 1,3-butariediol, 1,4-butanediol, 1,2-pentanediol, 1,5-pentanediol, 1,2-hexanediol, 1,6-hexanediol, 1,2-octanediol, 2-methylpentane-2,4-diol, or 1,2-decanediol; terpenes such as menthol, thymol, methone, carvacrol, or cineole; fatty acid esters such as isopropyl rnyristate, isopropyl palmitate, methyipropionate, ethyl oleate, or caprylic/capric triglyceride; combinations of any of the aforementioned classes; and combinations of any individual compounds within an aforementioned class. It will be appreciated that a person skilled in the art of topical drug delivery or transdermal drug delivery can select from the variety of skin penetration enhancers known in the art, both those listed here and other known enhancers. A review of enhancers is provided in Chantasart et anon., “Structure Enhancement Relationship of Chemical Penetration Enhancers in Drug Transport across the Stratum Corneum,” Pharmaceutics 2012, 4, 71-92; doi:10.3390/pharmaceutics 4010071. In certain inventive embodiments, the one or more skin penetration enhancers include a combination of at least two diols (e.g., a total of three diols), and optionally at least one additional skin penetration enhancer compound such as caprylic/capric triglyceride. For example, a combination of any two or three of the following diols may be used: 1,2-propanediol, 1,2-hexanediol, and 1,2-octanediol, with or without caprylic/capric triglyceride. Although it is contemplated that an alcohol may be included in the formulation as either a skin penetration enhancer (see above) or a solvent or co-solvent (see below), or both, the inventive formulations used in the present methods specifically exclude beer and other alcoholic beverages.

A skin penetration enhancer or combination of skin penetration enhancers is present in an inventive formulation in specific embodiments from 0.01 to 100 total weight percent. For example, the skin penetration enhancer or combination of skin penetration enhancers is present from 0.1 to 99 weight percent, or 0.1 to 70 weight percent, or 1 to 50 weight percent, or 1 to 30 weight percent, or 2 to 20 weight percent, or 3 to 20 weight percent, or 4 to 20 weight percent, or 5 to 20 weight percent, or 5 to 15 weight percent, or 5 to 12 weight percent, or 5 to 10 weight percent, or 5 to 8 weight percent. An individual skin penetration enhancer may be present from 0.001 to 100 weight percent, e.g., 0.5 percent, 1 percent, 2 percent, 3 percent, 4 percent, or 5 percent, whether or not one or more other skin penetration enhancers are present.

In some inventive embodiments, at least one of the skin penetration enhancers has a log K octanol/water partition coefficient of between 0.4 and 3. In still other embodiments, the log K octanol/water partition coefficient is between 0.5 and 2.1.

In some inventive embodiments, a solvent system is employed with the skin penetration enhancer, where the solvent system includes a majority solvent and a cosolvent. Factors relevant to selecting a solvent system include formulation viscosity, solubilization of skin penetration enhancer, and solubilization of other formulation additives.

Solvents operative herein are limited only by compatibility with skin and hair, and with the ingredients present in the formulation. Solvents operative herein illustratively include water and skin-compatible aqueous solutions of water-miscible cosolvents. Solvents or cosolvents operative herein illustratively include alcohols such as ethanol, propanols, and butanols; ketones such as acetone; and esters such as acetates. It is appreciated that several skin penetration enhancers, such as some diols and other alcohols, are able to solubilize other skin penetration enhancers or otherwise serve as a solvent. A given ingredient in a formulation of the invention may in some cases thus serve both as a skin penetration enhancer and as a solvent or cosolvent (and perhaps in other roles as well, such as a skin conditioner or a biocide). In such a situation, the ingredient may be categorized not only as a skin penetration enhancer, but also as solvent or cosolvent or whatever its other role(s) may be.

In addition to the skin penetration enhancer(s) as an active agent for retaining hair on the scalp of a subject, a variety of additives may be incorporated into the formulation. An additive may be included for a variety of reasons, illustratively including to facilitate subject compliance in application, to enhance the experience of formulation application, or to enhance shelf-stability. Additives operative herein illustratively include a pH buffer, an acid or base used to adjust pH, a biocide, an antioxidant, a surfactant, a fragrance, an astringent, or a combination thereof. Typically the additive does not itself promote hair retention or hair growth when applied to a scalp in the absence of the skin penetration enhancer, though it may function to improve the activity of the skin penetration enhancer somewhat.

pH buffers are commonly utilized in the field. A buffer can be an acid, a base, a salt of an acid, or a combination thereof utilized to equilibrate an aqueous solution to a desired pH. It is appreciated that pH measurement is routine, carried out via volumetric titration or with potentiometric electrodes such as those commercially available from Thermo Fisher Scientific (Waltham Mass., USA) or with litmus paper kits. pH buffering is readily achieved to maintain a pH range between 4 and 10 in certain inventive embodiments. In other inventive embodiments, the pH is adjusted to between 5 and 8, e.g., between 6 and 7.5. Buffers operative in the present invention illustratively include citrates such as alkali metal citrates; ascorbates; phosphates such as sodium phosphate; carbonates such as sodium bicarbonate; acids; and combinations thereof.

In some inventive embodiments, a biocide such as a broad spectrum biocide or a combination of biocides is present. A broad spectrum biocide is defined herein as having activity against Gram positive bacteria, Gram negative bacteria, and fungi, particularly those associated with the skin and hair of a healthy human subject. Biocides operative herein illustratively include C₄-C₁₆ alkanediols, benzoic acid, parabens, salicylic acid, carbolic acid, sorbic acid, alkyl p-hydroxybenzoates, p-chlorometacresol, hexachlorophene, benzalkonium chloride, chlorohexidine chloride, trichlorocarbanilide, phenoxyethanol, acylsarcosines, glutathione, malic acid, tartaric acid, ascorbic acid, ascorbates, ascorbyl glucoside, essential plant oils, mutacin proteins, and combinations thereof. In certain embodiments of the present invention, only naturally-derived biocides are present. Naturally-derived biocides illustratively include fermentation filtrates such as those of Lactobacillus, Streptococcus mutans, and Leuconostoc; bisabolol; eucalyptol; thymol; inositol; saponins; and natural extracts of plants, such as Japanese honeysuckle (Lonicera japonica), yangti (Rumex japonicus), kushen (Sophora flavescens), candock, wild oregano, orange, sage, manifoil, common mallow, chuanxiong (Cnidii officinale Makino), Japanese green gentian (Swertia japonica Makino), thyme, danguii (Angelica sinensis), orange peel, birch, field horsetail, dishcloth gourd, horse chestnut tree, creeping saxifrage (Saxifrage stolonifera), arnica, lily, mugwort, peony, aloe, and gardenia, as well as those detailed in M. M. Cowan, Clinical Microbiology Reviews, 12(4) October 1999, pages 564-582; or combinations thereof. Such extracts are obtained by procedures detailed in Clinical Microbiology Reviews, 12(4) October 1999, pages 573-574, often using a hydrophilic organic solvent (such as a C₁-C₈ alcohol or a polyhydric alcohol), water, or an aqueous alcohol. In specific inventive embodiments, Japanese honeysuckle, phloretin (apples), and galangin (Helichrysum aureonitens) are used as naturally derived biocides, each alone or in combination. It is appreciated that, in addition to biocide properties, a natural extract may impart a fragrance to an inventive formulation. A biocide is typically present in multi-use packages of an inventive formulation in specific embodiments from 0.1 to 10 total weight percent, though more than 10 total weight percent is readily included as desired—particularly where the biocide plays multiple roles in the formulation. It is appreciated that several skin penetration enhancers (e.g., some diols and terpenes) may act as biocides, so would fall into both categories.

In still other inventive embodiments, the formulation may include an antioxidant. Antioxidants operative herein illustratively include alpha lipoic acid, beta-glucan, coenzyme Q10, grape seed extract, oat extract, chamomile, green tea, soybean sterols, superoxide dismutase, vitamin C or a derivative thereof (such as ascorbic acid 2-glucoside, ascorbyl palmitate or magnesium ascorbyl palmitate), and vitamin E (alpha tocopherol or a tocotrienol), and combinations thereof.

In still other inventive embodiments, an inventive formulation includes an astringent. “Astringent” as used herein is defined as a substance having localized protein coagulant properties when applied topically to skin. Astringents operative herein illustratively include witch hazel, aluminum acetate, and aluminum sulfate, and combinations thereof. An astringent, when present, is typically in an amount of 0.01 to 5 total weight percent.

In certain embodiments, an inventive formulation is applied as a leave-in spray, gel, foam, or rinse. It is appreciated that aqueous foaming agents are conventional to the hair treatment industry and readily provided in an inventive formulation. In some embodiments, the formulation is not a shampoo or hair conditioner, and particularly not a hair conditioner that would be rinsed out within minutes of application to the scalp. In some embodiments, the formulation is a leave-in conditioner that normally would not be rinsed out until the next shampoo, e.g., at least 8 hours later.

In still other embodiments of the present invention, an inventive formulation is supplied in a sterile package, such as a multi-use spray bottle or can, or in single-use ampules. It is appreciated that radiation sterilization, heat sterilization, and size exclusion sterilization are suitable techniques, depending on the stability of the formulation ingredients to such techniques.

The inventive formulation may be provided in a vessel or other container that is equipped with, or at least packaged with, a device suitable for applying the formulation to a human scalp, the device being a spray pump (e.g., an atomizer pump), a sponge applicator, or an eye dropper. In some embodiments, the vessel or container is formed of metal, plastic or glass.

Exemplary inventive formulations are described in Tables 1 to 3.

TABLE 1
Typical ranges of ingredients in an inventive hair retentive
formulation, where percentages are total weight percent.

| | |
|---|---|
| Skin penetration enhancer(s) | 0.1 to 100% |
| Antioxidant | 0 to 5% |
| pH adjuster and/or buffer | 0 to 5% |
| Biocide | 0 to 10% |
| Fragrance | 0 to 5% |
| Astringent | 0 to 1% |
| Vitamins | 0 to 1% |
| Solvent system | Remainder |

TABLE 2
Exemplary formulation, where percentages
are total weight percent.

| | |
|---|---|
| Skin penetration enhancer(s) | 1 to 6% |
| Buffer | 0.01 to 0.5% |
| pH adjuster | (sufficient to adjust pH to 6.0-7.5) |
| Ascorbic acid derivative | 0.0001 to 0.01% |
| Biocide | 0.0001 to 0.01% |
| Water | Remainder |

TABLE 3
Exemplary formulation, where percentages
are total weight percent.

| | |
|---|---|
| Symdiol 68 ® (50:50 1,2-hexanediol and 1,2-octanediol) | 1% |
| Hydrolite-5 ® (1,2 pentanediol) | 5% |
| Kalpariane ® (caprylic/capric triglyceride | 0.001% |
| and Alaria Esculenta extract) | |
| Citric Acid | 0.015% |
| Sodium Citrate | 0.150% |
| NaOH (20%) (to adjust pH to 6.5-7.0) | 0.027% |
| Ascorbic Acid-2-Glucoside | 0.001% |
| Water | Remainder |

The formulation is used in a method of promoting hair retention on a human scalp. This method includes applying the formulation to the scalp of a subject at least once per week (e.g., at least twice per week or at least three times per week or at least once every other day or at least once per day) for at least four weeks, e.g., at least eight weeks or at least 12 weeks or at least 20 weeks, and potentially indefinitely for as long as the subject chooses to continue use. When a pump sprayer device (generally equipped with a conventional atomizer nozzle) is used, the operator sprays a mist of the liquid onto the subject's scalp. It may take multiple sprays from the pump (e.g., 3, 4, 5, or 6) to achieve overall treatment of the entire scalp. In general, a total of 0.1 to 5 ml of the formulation is sufficient for each overall application, with the amount needed depending on factors such as the concentration of active ingredient and the viscosity of the formulation. The formulation is typically a leave-in formulation, i.e., is left to dry on the scalp and is not immediately rinsed or washed out. The time between application of the formulation and the next shampoo is not critical, but ideally would be at least an hour.

The present invention is further illustrated with reference to the following non-limiting examples.

### Example 1

A formulation containing 5.0 total weight percent 1,2-pentanediol, 0.50 total weight percent 1,2-hexanediol, and 0.50 total weight percent 1,2-octanediol in deionized water was prepared.

### Example 2

A formulation containing 0.5 total weight percent 1,2-hexanediol and 0.5 total weight percent 1,2-octanediol in deionized water was prepared.

### Example 3

A formulation containing 5.0 total weight percent 1,2-pentanediol in deionized water was prepared.

### Example 4

Subjects were recruited for a clinical study testing the formulations of Examples 1-3, with enrollment criteria that included: the subject is female between the ages of 25 and 55 years; subject normally uses shampoo and conditioner; subject has hair length that is sufficient for evaluation, as determined by the cosmetologist; subject has Skin Type I-IV (ranging from white to moderately brown with skin reaction to sun within the range of “always burns” to “tans with ease”, respectively); subject sheds at least 20 hairs at the baseline evaluation; subject agrees to replace her usual hair shampoo and conditioner with the provided materials during the conditioning phase and for the duration of the study; subject agrees to apply the assigned scalp treatment once every other day for the duration of the study; subject agrees to protect the scalp from sun exposure; subject is in generally good health; and subject is dependable and able to follow directions as outlined in the protocol.

Enrolled subjects were instructed to apply the test material to the scalp every other day during the 12 week use period. Subjects were instructed to refrain from test material use and washing/conditioning the hair the morning of each visit. Subjects were also instructed to refrain from using any shampoos, conditioners, and scalp treatments other than the materials provided, but were told they may continue styling their hair in the usual manner (e.g., blow dry, flat iron, curling iron) and may use their usual styling products.

After approximately 4, 8, and 12 weeks of product use, subjects returned to the laboratory with unwashed hair, having refrained from test material application that day. At each visit, Mediscope® images of the scalp were obtained as described below. Visual grading of the hair was performed as described below. Wet and dry hair collections were performed as described.

Mediscope® combines the high resolution of digital photography with the direct capture features of a software archiving system and, in doing so, simplifies the photo-documentation process. Mediscope® utilizes a Canon Powershot® G10 (14.7 Mega Pixel) camera. This camera takes photos of outstanding quality that can be seen immediately on-screen. The whole imaging process is completely controlled by the software. An overlay feature allows the baseline images to be overlaid onto the live preview images, thus ensuring almost exact repositioning at every visit. One digital image of the scalp (crown of the head) was collected at each visit. The hair of each subject was assessed by a cosmetologist using a modified Visual Analog Scale (VAS) at each visit. The cosmetologist selected a location on the 100 mm VAS scale that corresponds with the assessment of hair thickness/volume, in relation to the labeled vertical positions on the scale. The distance between the mark recorded and the left origin of the line was subsequently measured in millimeters, to allow for assignment of a numerical score for the evaluated parameter.

At each visit to the laboratory, the subject's hair was shampooed/conditioned by the cosmetologist at the laboratory using Free & Clear™ Shampoo and Conditioner. Subjects wore a drape (white for dark-haired subjects and black for light-haired subjects). The hair was detangled with a comb or pick, if necessary. The hair was divided into five sections. Each section was combed from root to end for five strokes, using a wide tooth comb. Any hair shed onto the drape or in the sink was collected and counted. The hair was then finger-blown dry. Once the hair was dry, the hair was divided into five sections. Each section was combed from root to end for five strokes, using a wide tooth comb. Any hair shed onto the drape was collected and counted.

The study results are provided in Table 4, which reports mean total hair count of hair shed and collected at baseline, 4 weeks, 8 weeks, and 12 weeks. To maintain this favorable reduction in the rate of hair loss indefinitely, usage in perpetuity is contemplated.

TABLE 4
Total Hair Counts Shed

| Formula | Baseline | 4 weeks | 8 weeks | 12 weeks |
|---|---|---|---|---|
| Example 1 | 56 | 41 | 35 | 30 |
| Example 2 | 57 | 43 | 35 | 29 |
| Example 3 | 56 | 47 | 40 | 37 |

### Example 5

The formulation described in Table 3 was tested on a group of 31 subjects selected based on the same criteria as provided above in Example 4. Total hair counts shed at each visit (wet and dry combined) were determined. The study results are shown in Table 5.

TABLE 5
Total Hair Counts Shed After Use of Table 3 Formulation

| | 4 | 8 | 12 | 16 | 20 | 24 |
|---|---|---|---|---|---|---|
| Baseline | weeks | weeks | weeks | weeks | weeks | weeks |
| 64 | 49 | 47 | 36 | 37 | 30 | 31 |

Any patents or publications mentioned in this specification are herein incorporated by reference to the same extent as if each individual publication were explicitly and individually indicated to be incorporated by reference.

1. A method of treating a human subject to promote hair retention on the subject's scalp, the method comprising:
identifying a human subject in need of a reduction in rate of hair loss; and
applying topically to the scalp of the subject, at least once per week for at least four weeks, an effective amount of a formulation comprising one or more skin penetration enhancers that are effective in reducing the rate of hair loss when applied to a scalp,
wherein the one or more skin penetration enhancers are the only ingredients in the formulation that are active in reducing the rate of hair loss or otherwise increasing the number of hairs on a human scalp.

2. The method of claim 1, wherein the one or more skin penetration enhancers include a C₃-C₁₆ diol.

3. The method of claim 1, wherein the one or more skin penetration enhancers include 1,2-pentanediol.

4. The method of claim 1, wherein the one or more skin penetration enhancers include 1,2-hexanediol.

5. The method of claim 1, wherein the one or more skin penetration enhancers include 1,2-octanediol.

6. The method of claim 1, wherein the one or more skin penetration enhancers include at least two C₃-C₁₆ diols.

7. The method of claim 1, wherein the one or more skin penetration enhancers include at least three C₃-C₁₆ diols.

8. The method of claim 1, wherein the one or more skin penetration enhancers include 1,2-hexanediol and 1,2-octanediol.

9. The method of claim 1, wherein the one or more skin penetration enhancers include 1,2-pentanediol, 1,2-hexanediol, and 1,2-octanediol.

10. The method of claim 1, wherein each of the one or more skin penetration enhancers has a log K octanol/water partition coefficient of between 0.4 and 3.

11. The method of claim 1, wherein the one or more skin penetration enhancers include caprylic/capric triglyceride.

12. The method of claim 1, wherein the one or more skin penetration enhancers include 1,2-pentanediol, 1,2-hexanediol, 1,2-octanediol, and caprylic/capric triglyceride.

13. The method of claim 1, wherein the one or more skin penetration enhancers include a C₃-C₁₆ alkanol.

14. The method of claim 1, wherein the one or more skin penetration enhancers together constitute about 0.1 to about 20 total weight percent of the formulation.

15. The method of claim 1, wherein the one or more skin penetration enhancers together constitute about 1 to about 10 total weight percent of the formulation.

16. The method of claim 1, wherein the formulation comprises water.

17. The method of claim 1, wherein the formulation is applied at least as frequently as every other day for at least four weeks.

18. The method of claim 1, wherein the formulation is applied at least as frequently as every other day for at least eight weeks.

19. The method of claim 1, wherein the formulation is applied for at least 20 weeks.

20. The method of claim 1, wherein the formulation is applied in the form of a leave-in spray.

21. The method of claims 1, wherein the formulation is applied in the form of a leave-in foam or rinse.

22. The method of claim 1, wherein the method results in a greater than 20% reduction in the rate of hair loss, compared to the subject's rate of hair loss immediately prior to beginning use of the formulation.

23. The method of claim 1, wherein, if the formulation is applied to the scalp of each of a plurality of human test subjects for at least 20 weeks, the formulation is effective in producing a 20% or greater reduction in the average number of hairs shed by the test subjects at a point in time 20 weeks from day zero, compared to the average number of hairs shed by the test subjects on day zero.

24. The method of claim 1, wherein the formulation is not a shampoo or a hair conditioner.

25. A method of treating a human subject to promote hair retention on the subject's scalp, the method comprising
identifying a human subject in need of a reduction in rate of hair loss; and
applying topically to the scalp of the subject, at least once every two days for at least four weeks, an effective amount of a formulation that is an aqueous solution comprising an octanediol and a hexanediol, wherein the formulation does not contain proteins from hatching eggs.

26. The method of claim 25, wherein the octanediol is 1,2-octanediol and the hexanediol is 1,2-hexanediol, and each constitutes at least 0.5 total weight percent of the formulation.

27. The method of claim 25, wherein the formulation further comprises 1,2-pentanediol in an amount that constitutes at least 5 total weight percent of the formulation.

28. The method of claim 25, wherein the formulation contains no other ingredient active in reducing the rate of hair loss or otherwise increasing the number of hairs on a human scalp.

29. A product for reducing the rate of hair loss on the scalp of a human subject, the product comprising a vessel containing a formulation that is an aqueous solution comprising one, two or three diols selected from octanediols, hexanediols, and pentanediols, the diol(s) being present in the formulation in an amount that promotes hair retention when applied to a human scalp,
wherein the formulation is suitable for topical application to a human scalp and contains no proteins and no active ingredients other than the diol(s), and
wherein the product comprises a device suitable for applying the formulation to a human scalp, the device being a spray pump, a sponge applicator, or an eye dropper.

30. The product of claim 29, wherein the formulations comprises at least 0.5 total weight percent 1,2-octanediol and at least 0.5 total weight percent 1,2-hexanediol.
